SEQUENCE LISTING (4 sequences)

SEQ ID NO: 1 - protein, 102 aa, Dengue virus
SYSMCTGKFKVVKEIAETQHGTIVIRVQYEGDGSPCKIPFEIMDLEKRHVLGRLITVNPIVTEKDRPVNIEAEPPFGDSYIIIGVEPGQLKLNWFKKGSSIG

SEQ ID NO: 2 - protein, 428 aa, Geobacillus stearothermophilus
MAFEFKLPDIGEGIHEGEIVKWFVKPGDEVNEDDVLCEVQNDKAVVEIPSPVKGKVLEILVPEGTVATVGQTLITLDAPGYENMTFKGQEQEEAKKEEKTETVSKEEKVDAVAPNAPAAEAEAGPNRRVIAMPSVRKYAREKGVDIRLVQGTGKNGRVLKEDIDAFLAGGAKPAPAAAEEKAAPAAAKPATTEGEFPETREKMSGIRRAIAKAMVHSKHTAPHVTLMDEADVTKLVAHRKKFKAIAAEKGIKLTFLPYVVKALVSALREYPVLNTSIDDETEEIIQKHYYNIGIAADTDRGLLVPVIKHADRKPIFALAQEINELAEKARDGKLTPGEMKGASCTITNIGSAGGQWFTPVINHPEVAILGIGRIAEKPIVRDGEIVAAPMLALSLSFDHRMIDGATAQKALNHIKRLLSDPELLLMEA

SEQ ID NO: 3 - DNA, 35 bp, Dengue virus
  misc_feature (1)..(4): n is a, c, g, or t
nnnnccatgccgatgtcatactctatgtgcacagg

SEQ ID NO: 4 - DNA, 35 bp, Dengue virus
  misc_feature (1)..(4): n is a, c, g, or t
nnnncccggggccgatagaacttcctttcttaaac

(19) United States
(12) Patent Application Publication
(10) Pub. No.: US 2017/0143817 A1
(43) Pub. Date: May 25, 2017

(54) DENGUE VIRUS VACCINE

(71) Applicants: William Messer, Portland, OR (US); Nancy Haigwood, Beaverton, OR (US); Sean P. McBurney, Portland, OR (US)

(72) Inventors: William Messer, Portland, OR (US); Nancy Haigwood, Beaverton, OR (US); Sean P. McBurney, Portland, OR (US)

(73) Assignee: OREGON HEALTH & SCIENCE UNIVERSITY, PORTLAND, OR (US)

(21) Appl. No.: 15/357,608

(22) Filed: Nov. 21, 2016

Related U.S. Application Data
(60) Provisional application No. 62/257,881, filed on Nov. 20, 2015.

(51) Int. Cl. A61K 39/12; A61K 9/14; C12N 7/00; C07K 14/005

(52) U.S. Cl. CPC A61K 39/12; C07K 14/005; A61K 9/14; C12N 7/00; A61K 2039/54

(57) ABSTRACT

Recombinant fusion proteins including a dengue virus EIII and an E2 subunit derived from a thermophilic bacterium are described. Also described are expression vectors including a polynucleotide that encodes the recombinant fusion protein and a promoter. Also described are vaccine compositions that include either the recombinant fusion protein, polynucleotide, or both. Also described are methods of generating an immune response to dengue virus serotype 2 comprising administering one or more of the disclosed vaccine compositions.

### RELATED APPLICATIONS

This application claims the benefit of U.S. Provisional Patent Application 62/257,881, entitled DENGUE VIRUS VACCINE, filed 20 Nov. 2015, and which is incorporated by reference herein.

### ACKNOWLEDGEMENT OF GOVERNMENT SUPPORT

The disclosed invention was created with the support of the United States government under the terms of grant numbers P51 OD011092, UL1 TR000128, RO1 AI074379, and F32 A1106489, all awarded by the National Institutes of Health. The United States government has certain rights to this invention.

### FIELD

Generally, the field is vaccines. More specifically the field is vaccines that protect against Dengue virus infection

### BACKGROUND

Dengue virus (DENV) is the most significant arthropod borne virus in the world. There are currently over three billion people at risk for dengue virus infection, with an estimated 200 million individuals infected every year (Bhatt S et al, Nature 496, 504-507 (2013); incorporated by reference herein). While a majority of DENV infections are asymptomatic or result in dengue fever-characterized by high fever and severe joint/muscle pain, DENV infection can also cause severe dengue disease, which can lead to hospitalization and/or death. It is estimated that there are over 100,000 cases of severe dengue annually, leading to more than 20,000 deaths per year. The economic impact of dengue infection is estimate to be $1-4 billion per year (Shepard D S et al, PLoS Negl Trop Dis 8, e3306 (2014); incorporated by reference herein). Given the global burden of DENV disease, there is an urgent need for a DENV vaccine. Although none is currently available, several are under development. Analyses of phase 3 trials of the most advanced DENV vaccine to date, the Sanofi CYD-TDV live-attenuated vaccine (LAV), highlight both the promise and challenges of current LAV approaches. Pooled results (Hadinegoro S R, N Engl J Med 373, 1195-1206 (2015); incorporated by reference herein) of three separate trials (Villar L et al, N Engl J Med 372, 113-123 (2015); Capeding M R et al, Lancet 384, 1358-1365 (2014); and Sabchareon A et al, Lancet 380, 1559-1567 (2012); all of which are incorporated by reference herein) show an age-dependent protective effect. Overall vaccine efficacy was 67.8% against all serotypes for vaccinees aged >9 years old and 44.6% for vaccinees <9 years old. Moreover, this protection was biased towards vaccinees in both age groups who were DENV seropositive before first vaccine dose. Additionally, while the vaccine significantly protected against hospitalization with DENV, this protection was primarily in vaccinees aged >9 years old, and risk of hospitalization for severe dengue was elevated in vaccines aged <9 years old. Finally, serotype specific protection varied substantially, from 47.1% against DENV-2 to 81.9% against DENV-4 for vaccinees aged >9 and 33.6% for DENV-2 and 62.1% against DENV-3 for vaccinees aged <9 yrs. These variable results are not fully understood, but are hypothesized to be related to limited immunogenicity of the vaccine strains, which is hypothesized to be driven by serotype immunodominance and LAV competition, particularly in DENV naïve recipients.

### SUMMARY

Disclosed herein is the development of an alternate approach to LAVs, using protein scaffold and DNA-based DENV vaccine targeting domain III of the E glycoprotein (EDIII). The DENV E glycoprotein exists as homo-dimers with 3 distinct domains—I, II, and Ill, that, on the mature DENV virion, are arranged in a flat herringbone pattern with icosahedral symmetry (Kuhn R J et al, Cell 108, 717-725 (2002); incorporated by reference herein). Domains I and II are linearly discontinuous and fold to form a central eight-stranded β barrel (domain I) with a lateral protrusion (domain II) that contains the highly conserved fusion loop required for virion fusion with endosomes. Domain III (EDIII) is a continuous peptide that extends from domain I and forms an Ig like fold that is believed to be the ligand for an as yet unidentified cellular receptor. When expressed as a recombinant polypeptide, EDIII preferentially folds into its native conformation (Wahala W M et al, Virology 392, 103-113 (2009); incorporated by reference herein) and EDIII has been shown to elicit potently neutralizing antibodies that target tertiary epitopes displayed on wild-type virus (Beltramello M et al, Cell Host Microbe 8, 271-283 (2010) and Guzman M G et al, Expert Rev Vaccines 9, 137-147 (2010); both of which are incorporated by reference herein). For these reasons, EDIII was thought to be an excellent candidate antigen for incorporation in a protein scaffold vaccine, due to its immunogenicity and the fact that no molecular engineering would be required to maintain epitope fidelity. Multiple reports of DENV EDIII based recombinant protein vaccines have been published in the past 10 years, but only a handful have undergone immunogenicity trials in non-human primates (Suzarte E et al, Int Immunol 27, 367-379 (2015); Gil L et al, Immunol Cell Biol 93, 57-66 (2015); Chen H W et al, Arch Virol 158, 1523-1531 (2013); Bernardo L et al, Clin Vaccine Immunol 16, 1829-1831 (2009); Bernardo L et al, Arch Virol 153, 849-854 (2008); Valdes I et al, Vaccine 27, 995-1001 (2009); Liu G et al, Clin Vaccine Immunol 22, 516-525 (2015); Bernardo L et al, Antiviral Res 80, 194-199 (2008); and Hermida L et al, Vaccine 24, 3165-3171 (2006); all of which are incorporated by reference herein) with even fewer evaluating protection on challenge, with mixed results, and the threshold of protection for EDIII based vaccines remains poorly defined.

For the study described here, recombinant DENV-2 EDIII was presented on the surface of the E2 protein, which is a subunit of the pyruvate dehydrogenase (PDH) complex from Geobacillus stearothermophilus that self assembles into a 60-mer particle. In the native enzyme complex, the N-terminus of each E2 subunit associates non-covalently with 60 copies of E1 (150 kDa) or E3 (100 kDa) enzymes (Lessard I A & Perham R N, Biochem J, 306, 727-733 (1995). Using the E2 display (E2DISP) expression system (Domingo G J et al, J Mol Biol 305, 259-267 (2001) and Domingo G J et al, Eur J Biochem 266, 1136-1146 (1999); both of which are incorporated by reference herein), up to 60 polypeptides can be presented on the E2 scaffold as N-terminal fusion proteins without negatively impacting the native folding of the E2 core. Sixty E2 monomers self-assemble into a pentagonal dodecahedral scaffold with icosahedral symmetry, forming a large multimeric particle with a molecular weight >1.5 MD and a diameter of ^{˜}24 nm. Like many proteins from thermophilic bacteria, E2 is heat stable and can be renatured in vitro from denaturing conditions to form the 60-mer scaffold without the need of chaperonins. E2 can be modified at the N-terminus by replacing the natural E2 peripheral domains with exogenous polypeptides, creating a novel E2 multimeric antigen display system. Multimeric E2 protein scaffolds as a vaccine platform to display regions of HIV-1 Gag and Env have been described (Caivano A et al, Virology 407, 296-305 (2010); Jaworski J P et al, PLoS One 7, e31464 (2012); and Krebs S J et al, PLoS One 9, e113463 (2014); all of which are incorporated by reference herein). The multimeric particle may also improve immunogenicity by providing bivalent binding opportunities for Abs, and for DENV presents the opportunity to display EDIII from all four serotypes in a single particle. E2-based scaffolds lack viral genetic material or enzymes and thus are inherently safer than attenuated or inactivated wild-type viruses. The scaffold protein is co-delivered with EDIII DNA in an expression plasmid.

Described herein are the immunogenicity and protection conferred by co delivery of an EDIII-E2 protein particle and EDIII expression plasmid into flavivirus-naïve rhesus macaques. Both vaccine components utilized the EDIII region of DENV2 isolate 16681. The Gene Gun delivered DNA component has previously been shown to augment the humoral immune response following vaccination. The EDIII-E2 vaccine was both highly immunogenic and conferred protection upon challenge with the heterologous DENV-2 16803, demonstrating that a DNA and protein scaffold-based DENV vaccine is a viable alternative to current DENV vaccine strategies.

Disclosed herein is a recombinant protein comprising a first polypeptide comprising a dengue virus E glycoprotein domain III and a second polypeptide comprising an E2 subunit of a pyruvate dehydrogenase from a thermophilic bacterium. The E glycoprotein domain III can be derived from a dengue virus of serotype DENV-2. The E2 subunit can be derived from Geobacillus stearothermophilus.

Disclosed herein is an expression vector comprising a protein that encodes the recombinant polypeptide described above operably linked to a promoter.

Disclosed herein is a pharmaceutical composition comprising the recombinant polypeptide and/or the expression vector described above. The pharmaceutical composition can further comprise an adjuvant.

Disclosed herein is a method of generating an immune response to dengue virus in a subject comprising administering an effective amount of the pharmaceutical composition described above to the subject. The immune response can further comprise a protective immune response.

Disclosed herein is a recombinant fusion protein that includes a first polypeptide comprising SEQ ID NO: 1 and a second polypeptide comprising SEQ ID NO: 2. The first polypeptide is located N-terminal to the second polypeptide.

Disclosed herein is an expression vector that includes a polynucleotide that encodes the recombinant fusion protein of claim 1 and a promoter, where the promoter is operably linked to the polynucleotide. In embodiments, the polynucleotide can be derived from amplifying a nucleic acid fragment from dengue virus serotype 2 strain 16681 using oligonucleotides comprising SEQ ID NO: 3 and SEQ ID NO: 4.

Disclosed herein are vaccine compositions that include the recombinant fusion described above and/or the expression vector described above. The vaccine composition can further comprise an adjuvant. The vaccine composition can further comprise 1 μm diameter gold beads, particularly if it includes the expression vector.

Disclosed herein are methods of generating an immune response to dengue virus serotype 2 in a subject. The methods involve administering an effective amount of the vaccine composition described above to the subject. The immune response can be a protective immune response. If the vaccine composition comprises the recombinant fusion protein described above, the pharmaceutical composition can be administered intramuscularly. If the vaccine composition comprises the expression vector described above, then the pharmaceutical composition can be administered intradermally.

In an embodiment, the method involves administering both the pharmaceutical composition that includes the recombinant fusion protein and the pharmaceutical composition that includes the expression vector to the subject. In such an embodiment, the recombinant fusion protein can be administered at a dosage of between 65 and 95 μg/kg and the expression vector can be administered at between 5 μg/kg and 7 μg/kg. In such an embodiment, the two pharmaceutical compositions can be administered on the same day in a first joint administration. In such an embodiment, the two pharmaceutical compositions can be administered in a second joint administration following the first joint administration and again in a third joint administration 12 weeks following the first joint administration.

### BRIEF DESCRIPTION OF THE SEVERAL VIEWS OF THE DRAWINGS

Color figures were submitted as part of the original disclosure and references to colors in the figures are provided in this description. Applicants reserve the right to present color versions of the figures disclosed herein in later proceedings.

FIG. 1A: Transformed E. coli containing the EDIII-E2 expression plasmid were induced and lysed. Protein lysates were loaded into SDS-Page gels for visualization. The top panel shows E2 pre-induction. The second panel from the top shows E2 induced. The third panel from the top shows EDIII-E2 pre-induction. The bottom panel shows EDIII-E2 induced. One gel was stained for protein. Matching gels were transferred to nitrocellulose and probed by western blot for E2 (red) and EDIII (green), A merged figure is shown at the bottom of a showing the localization of both the E2 and EDIII stains.

FIG. 1B is an image of a blot showing purified EDIII-E2 particles separated by SDS-PAGE and then stained for protein. A previously purified EDIII-E2 sample is shown in lane 1. Lanes 3 and 4 show column fractions that were combined for the overall vaccination component.

FIG. 1C is an image of a gel showing that 293t cells were transformed with the EDIII vaccine DNA plasmid component eluted from vaccination bullets. Lane 1 shows an EDIII-E2 particle as a control. The remaining lanes are the supernatant and lysate from transfected cells following induction of DNA expression.

FIG. 2A is a plot showing 8A5 tested by surface plasmon resonance at the indicated concentrations. Binding is shown as RUs over injection time.

FIG. 2B Antibodies DVC 3.7, DVC 10.16, and DVC 14.2 were tested 3 times at 33.3 nM. Results are shown as Max RUs from each run.

For FIGS. 2A and 2B: EDIII-E2 and E2 only particles were immobilized on the surface of a Biacore® chip. Conformational antibodies were flowed over the surface of the chip to determine the availability of antibody binding sites.

FIG. 3A is a plot of antibody responses induced by EDIII-E2/EDIII DNA vaccination. Antibody responses were determined from serum samples obtained at the time of the first vaccination, and then two weeks following each vaccination. Dilutions of serum samples were tested for binding to E2 by ELISA. Results are reported as endpoint titer.

FIG. 3B is a plot of antibody responses induced by E DNA vaccination. Antibody responses were determined from serum samples obtained at the time of the first vaccination, and then two weeks following each vaccination. Dilutions of serum samples were tested for binding to E2 by ELISA. Results are reported as endpoint titer

FIG. 3C is a plot showing dilutions of serum samples were also tested for neutralizing antibodies by FRNT assay against matched DENV2 16681 virus(C). Results are reported as FRNT₅₀ titer.

FIG. 4 is a plot of neutralizing antibody responses were determined by FRNT assay against DENV-1 (WestPac '74), DENV-2 (16681), DENV-3 (UNC3001), and DENV-4 (TVP-360) viruses.

FIG. 5A is a plot showing the results whereby macaques were challenged with DENV2 16803. Serum samples were collected daily from day 0-10 and were tested for the presence of viral RNA by rtPCR. All macaques that were positive by rtPCR were further tested by qPCR to determine viral RNA levels.

FIG. 5B is a plot showing the results where macaques were tested for the presence of neutralizing antibodies to 16803 by FRNT assay.

FIG. 5C is a plot showing the results of vaccinated macaques tested at the time of viral challenge as well as 21 days post-challenge. All control macaques were tested at 21 days post-challenge. The fold change in FRNT₅₀ titer for vaccinated macaques was determined by dividing the day 21 titer by the titer at day 0.

FIG. 6 is a plot of the FRNT₅₀ Protective Threshold. Week 14 FRNT₅₀ titers against DENV2 16681 were compared between those vaccinated macaques that became viral RNA positive and those that remained protected. All macaques with FRNT₅₀ titers of greater than 1:25,000 were sterilely protected against DENV2 16803 viral challenge.

### SEQUENCE LISTING

SEQ ID NO: 1 is a polypeptide sequence of a dengue virus serotype 2 EIII domain.

SEQ ID NO: 2 is a polypeptide sequence of a Geobacillus stearothermophilus E2.

SEQ ID NO: 3 is an oligonucleotide primer used in amplifying a polynucleotide encoding SEQ ID NO: 1

SEQ ID NO: 4 is an oligonucleotide primer used in amplifying a polynucleotide encoding SEQ ID NO: 1.

### DETAILED DESCRIPTION

### I. Definitions

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCR Publishers, Inc., 1995 (ISBN 1-56081-569-8).

All publications, patents, patent applications, internet sites, and accession numbers/database sequences (including both polynucleotide and polypeptide sequences) cited herein are hereby incorporated by reference in their entirety for all purposes to the same extent as if each individual publication, patent, patent application, internet site, or accession number/database sequence were specifically and individually indicated to be so incorporated by reference.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below.

As will be understood by one of ordinary skill in the art, each embodiment disclosed herein can comprise, consist essentially of or consist of its particular stated element, step, ingredient or component. Thus, the terms “include” or “including” should be interpreted to recite: “comprise or consist of.” The transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified.

In addition, the materials, methods, and examples are illustrative only and not intended to be limiting. In order to facilitate review of the various embodiments of the disclosure, the following explanations of specific terms are provided:

Antigen: As used herein, the terms “antigen” or “immunogen” are used interchangeably to refer to a substance, typically a protein, which is capable of inducing an immune response in a subject. The term also refers to proteins that are immunologically active in the sense that once administered to a subject (either directly or by administering to the subject a nucleotide sequence or vector that encodes the protein) is able to evoke an immune response of the humoral and/or cellular type directed against that protein.

Administration: To provide or give a subject an agent, such as a composition comprising an effective amount of one or more of the disclosed antigens or a viral expression vector that expresses the disclosed antigen by any effective route. Exemplary routes of administration include, but are not limited to, injection (such as subcutaneous, intramuscular, intradermal, intraperitoneal, and intravenous), oral, sublingual, rectal, transdermal, intranasal, vaginal and inhalation routes. Multiple pharmaceutical compositions can be administered simultaneously, on the same day, or on different days, each via a different route of administration. For example, a first vaccine composition can be administered intramuscularly on the same day as a second vaccine composition administered intradermally.

Conservative variants: A substitution of an amino acid residue for another amino acid residue having similar biochemical properties. “Conservative” amino acid substitutions are those substitutions that do not substantially affect or decrease an activity of an MHC Class II polypeptide, such as an MHC class II al polypeptide. A polypeptide can include one or more amino acid substitutions, for example 1-10 conservative substitutions, 2-5 conservative substitutions, 4-9 conservative substitutions, such as 1, 2, 5 or 10 conservative substitutions. Specific, non-limiting examples of a conservative substitution include the following:

| Original Amino Acid | Conservative Substitutions |
|---|---|
| Ala | Ser |
| Arg | Lys |
| Asn | Gln, His |
| Asp | Glu |
| Cys | Ser |
| Gln | Asn |
| Glu | Asp |
| His | Asn, Gln |
| Ile | Leu, Val |
| Leu | Ile, Val |
| Lys | Arg, Gln, Glu |
| Met | Leu, Ile |
| Phe | Met, Leu, Tyr |
| Ser | Thr |
| Thr | Ser |
| Trp | Tyr |
| Tyr | Trp, Phe |
| Val | Ile, Leu |

Domain: A discrete part of an amino acid sequence of a polypeptide or protein that can be equated with a particular function.

Effective amount: As used herein, the term “effective amount” refers to an amount of an agent, such as a dengue virus vaccine, that is sufficient to generate a desired response, such as reduce or eliminate a sign or symptom of a condition or disease or induce an immune response to an antigen. In some examples, an “effective amount” is one that treats (including prophylaxis) one or more symptoms and/or underlying causes of any of a disorder or disease. An effective amount can be a therapeutically effective amount, including an amount that prevents one or more signs or symptoms of a particular disease or condition from developing, such as one or more signs or symptoms associated with infectious disease, cancer, or autoimmune disease.

Operably Linked: A promoter or other activating or suppressing nucleic acid sequence is operably linked with a polynucleotide when the promoter is placed in such a way that it has an effect upon the polynucleotide. For instance, a promoter is operably linked to a coding sequence if the promoter affects the transcription or expression of the coding sequence. Operably linked DNA sequences may be contiguous, or they may operate at a distance.

Pharmaceutically acceptable carriers: The pharmaceutically acceptable carriers of use are conventional. Remington's Pharmaceutical Sciences, by E. W. Martin, Mack Publishing Co., Easton, Pa., 19th Edition, 1995, describes compositions and formulations suitable for pharmaceutical delivery of the compositions disclosed herein. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (such as powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Polynucleotide: As used herein, the term “polynucleotide” refers to a polymer of ribonucleic acid (RNA) or deoxyribonucleic acid (DNA). A polynucleotide is made up of four bases; adenine, cytosine, guanine, and thymine/uracil (uracil is used in RNA). A coding sequence from a nucleic acid is indicative of the sequence of the protein encoded by the nucleic acid.

Polypeptide: The terms “protein”, “peptide”, “polypeptide”, and “amino acid sequence” are used interchangeably herein to refer to polymers of amino acid residues of any length. The polymer can be linear or branched, it may comprise modified amino acids or amino acid analogs, and it may be interrupted by chemical moieties other than amino acids. The terms also encompass an amino acid polymer that has been modified naturally or by intervention; for example disulfide bond formation, glycosylation, lipidation, acetylation, phosphorylation, or any other manipulation or modification, such as conjugation with a labeling or bioactive component.

Promoter: A promoter can be any of a number of nucleic acid control sequences that directs transcription of a nucleic acid. Expression by a promoter may be further modulated by enhancer or repressor elements. Numerous examples of promoters are available and well known to those of skill in the art. A nucleic acid comprising a promoter operably linked to a nucleic acid sequence that codes for a particular polypeptide can be termed an expression vector. An expression vector comprising a constitutively active promoter expresses the protein at effectively all times in the cell. A conditionally active promoter directs expression only under certain conditions. For example, a conditionally active promoter might direct expression only in the presence or absence of a particular compound such as a small molecule, amino acid, nutrient, or other compound while a constitutively active promoter directs expression independently of such conditions. Tissue specific promoters (as well as tissue specific enhancers and suppressors) activate or suppress expression in a particular tissue type.

Recombinant: A recombinant polynucleotide or polypeptide is one that has a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques. A recombinant polypeptide can also refer to a polypeptide that has been made using recombinant nucleic acids, including recombinant nucleic acids transferred to a host organism that is not the natural source of the polypeptide.

Sequence identity/similarity: The identity/similarity between two or more nucleic acid sequences, or two or more amino acid sequences, is expressed in terms of the identity or similarity between the sequences. Sequence identity can be measured in terms of percentage identity; the higher the percentage, the more identical the sequences are. Sequence similarity can be measured in terms of percentage identity or similarity (which takes into account conservative amino acid substitutions); the higher the percentage, the more similar the sequences are. Polypeptides or protein domains thereof that have a significant amount of sequence identity and also function the same or similarly to one another (for example, proteins that serve the same functions in different species or mutant forms of a protein that do not change the function of the protein or the magnitude thereof) can be called “homologs.” Methods of alignment of sequences for comparison are well known in the art. Various programs and alignment algorithms are described in: Smith & Waterman, Adv Appl Math 2, 482 (1981); Needleman & Wunsch, J Mol Biol 48, 443 (1970); Pearson & Lipman, Proc Natl Acad Sci USA 85, 2444 (1988); Higgins & Sharp, Gene 73, 237-244 (1988); Higgins & Sharp, CABIOS 5, 151-153 (1989); Corpet et al, Nuc Acids Res 16, 10881-10890 (1988); Huang et al, Computer App Biosci 8, 155-165 (1992); and Pearson et al, Meth Mol Bio 24, 307-331 (1994). In addition, Altschul et al, J Mol Biol 215, 403-410 (1990), presents a detailed consideration of sequence alignment methods and homology calculations.

The NCBI Basic Local Alignment Search Tool (BLAST) (Altschul et al, (1990) supra) is available from several sources, including the National Center for Biological Information (NCBI, National Library of Medicine, Building 38A, Room 8N805, Bethesda, Md. 20894) and on the Internet, for use in connection with the sequence analysis programs blastp, blastn, blastx, tblastn and tblastx. Additional information can be found at the NCBI web site. BLASTN is used to compare nucleic acid sequences, while BLASTP is used to compare amino acid sequences. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a nucleic acid sequence that has 1166 matches when aligned with a test sequence having 1154 nucleotides is 75.0 percent identical to the test sequence (1166÷1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer. In another example, a target sequence containing a 20-nucleotide region that aligns with 20 consecutive nucleotides from an identified sequence as follows contains a region that shares 75 percent sequence identity to that identified sequence (that is, 15÷20*100=75).

For comparisons of amino acid sequences or nucleotide sequences of greater than about 30 amino acids, the Blast 2 sequences function is employed using the default BLOSUM62 matrix set to default parameters, (gap existence cost of 11, and a per residue gap cost of 1). Homologs are typically characterized by possession of at least 70% sequence identity counted over the full-length alignment with an amino acid sequence using the NCBI Basic Blast 2.0, gapped blastp with databases such as the nr database, swissprot database, and patented sequences database. Queries searched with the blastn program are filtered with DUST (Hancock & Armstrong, Comput Appl Biosci 10, 67-70 (1994.) Other programs use SEG. In addition, a manual alignment can be performed. Proteins with even greater similarity will show increasing percentage identities when assessed by this method, such as at least about 75%, 80%, 85%, 90%, 95%, 98%, or 99% sequence identity to a protein.

When aligning short peptides or short oligonucleotides (fewer than around 30 amino acids), the alignment is be performed using the Blast 2 sequences function, employing the PAM30 matrix set to default parameters (open gap 9, extension gap 1 penalties). Proteins with even greater similarity to the reference sequence will show increasing percentage identities when assessed by this method, such as at least about 60%, 70%, 75%, 80%, 85%, 90%, 95%, 98%, or 99% sequence identity to a protein. When less than the entire sequence is being compared for sequence identity, homologs will typically possess at least 75% sequence identity over short windows of 10-20 amino acids, and can possess sequence identities of at least 85%, 90%, 95% or 98% depending on their identity to the reference sequence. Methods for determining sequence identity over such short windows are described at the NCBI web site. One indication that two nucleic acid molecules are closely related is that the two molecules hybridize to each other under stringent conditions, as described above. Nucleic acid sequences that do not show a high degree of identity may nevertheless encode identical or similar (conserved) amino acid sequences, due to the degeneracy of the genetic code. Changes in a nucleic acid sequence can be made using this degeneracy to produce multiple nucleic acid molecules that all encode substantially the same protein. Such homologous nucleic acid sequences can, for example, possess at least about 50%, 60%, 70%, 80%, 90%, 95%, 98%, or 99% sequence identity to a nucleic acid that encodes a protein.

Subject: As used herein, the term “subject” refers to a living multi-cellular vertebrate organism, a category that includes both human and non-human mammals, including non-human primates.

Treatment: As used herein, the term “treatment” refers to an intervention that ameliorates a sign or symptom of a disease or pathological condition. As used herein, the terms “treatment”, “treat” and “treating,” with reference to a disease, pathological condition or symptom, also refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, a reduction in the number of relapses of the disease, an improvement in the overall health or well-being of the subject, or by other parameters well known in the art that are specific to the particular disease. A prophylactic treatment is a treatment administered to a subject who does not exhibit signs of a disease or exhibits only early signs, for the purpose of decreasing the risk of developing pathology. A therapeutic treatment is a treatment administered to a subject after signs and symptoms of the disease have developed.

### Flaviviruses

Disclosed herein are vaccine compositions useful in the prevention of infection with a dengue virus. Dengue virus is an example of a flavivirus. Flaviviruses are small, enveloped viruses containing a single, positive-strand, genomic RNA, approximately 10,500 nucleotides in length containing short 5′ and 3′ non-translated regions (NTRs), a single long open reading frame, a 5′ cap, and a nonpolyadenylated 3′ terminus. Examples of flaviruses include all four dengue serotypes (DENV-1, DENV-2, DENV-3, and DENV-4) yellow fever virus, Japanese encephalitis virus, West Nile virus and tick-borne encephalitis virus and others. Flaviviral proteins are derived from a single polypeptide processed by host and viral proteases. The gene products encoded by the single open reading frame include capsid (C), preMembrane (prM, which is processed to Membrane (M) just prior to virion release from the cell), Envelope (E) and the non-structural (NS) proteins.

The dengue virus species comprises a variety of genetically distinct strains within four antigenically distinguishable serotypes known as DENV-1, DENV-2, DENV-3, and DENV-4. Dengue is a member of the genus Flavivirus in the family Flaviviridae (Calisher C H et al, J Gen Virol 70 (Pt1), 37-43 (1989); Rico-Hesse R, Virology 174, 479-493 (1990); and Weaver S C and Vasilakis N, Infect Genet Evol 9, 523-540 (2009); all of which are hereby incorporated by reference in their entireties.) Symptoms of DENV infection by any serotype may include but need not be limited to high fever, headache, aching muscles and joints, rash, severe hemorrhage, vascular permeability, shock, or any combination of these. The more severe form of dengue disease may be termed dengue hemorrhagic fever/dengue shock syndrome.

### Treatment of Flavivirus Infection:

Disclosed herein include methods of prophylactic treatment of a subject that may develop a flavivirus infection. The methods involve administering a vaccine composition that includes the disclosed protein composition to the subject.

A subject may be any multi-cellular vertebrate organisms, a category that includes human and non-human mammals, such as mice. In some examples a subject is a male. In some examples a subject is a female. Further types of subjects to which the pharmaceutical composition may be properly administered include subjects known to have a flavivirus infection (through, for example, a molecular diagnostic test or clinical diagnosis,) subjects having a predisposition to contracting a flavivirus infection (for example by travel to a region in which a flavivirus is endemic), or subjects displaying one or more symptoms of having a flavivirus infection.

Administration of an agent may be any method of providing or give a subject an agent, such as a vaccine composition protective against flavivirus infection, by any effective route. Exemplary routes of administration include, but are not limited to, injection (such as subcutaneous, intramuscular, intradermal, intraperitoneal, and intravenous), oral, sublingual, rectal, transdermal, intranasal, vaginal and inhalation routes.

Treating a subject may be any therapeutic intervention that ameliorates a sign or symptom of a disease or pathological condition after it has begun to develop, whether or not the subject has developed symptoms of the disease. Ameliorating, with reference to a disease, pathological condition or symptom refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, a reduction in the number of relapses of the disease, an improvement in the memory and/or cognitive function of the subject, a qualitative improvement in symptoms observed by a clinician or reported by a patient, or by other parameters well known in the art that are specific to flavivirus infection.

A symptom may be any subjective evidence of disease or of a subject's condition, for example, such evidence as perceived by the subject; a noticeable change in a subject's condition indicative of some bodily or mental state. A sign may be any abnormality indicative of disease, discoverable on examination or assessment of a subject. A sign is generally an objective indication of disease.

A pharmaceutical composition may be any chemical compound or composition capable of inducing a desired therapeutic or prophylactic effect when properly administered to a subject. A pharmaceutical composition can include a therapeutic agent, a diagnostic agent or a pharmaceutical agent. A therapeutic or pharmaceutical agent is one that alone or together with an additional compound induces the desired response (such as inducing a therapeutic or prophylactic effect when administered to a subject). In a particular example, a pharmaceutical agent is an agent that significantly reduces one or more symptoms associated with flavivirus infection. A pharmaceutical composition may be a member of a group of compounds. Pharmaceutical compositions may be grouped by any characteristic including chemical structure and the molecular target they affect.

The administration of vaccines for flavivirus infection can be for either prophylactic or therapeutic purposes. When provided prophylactically, the treatments for flavivirus infection are provided in advance of any clinical symptom of flavivirus infection. Prophylactic administration serves to prevent or ameliorate any subsequent disease process. When provided therapeutically, the compounds are provided at (or shortly after) the onset of a symptom of disease. For prophylactic and therapeutic purposes, the treatments for flavivirus infection can be administered to the subject in a single bolus delivery, via continuous delivery (for example, continuous transdermal, mucosal or intravenous delivery) over an extended time period, or in a repeated administration protocol (for example, by an hourly, daily or weekly, repeated administration protocol). The therapeutically effective dosage of the treatments for flavivirus infection can be provided as repeated doses within a prolonged prophylaxis or treatment regimen that will yield clinically significant results to alleviate one or more symptoms or detectable conditions associated with flavivirus infection.

A pharmaceutically acceptable carrier (interchangeably termed a vehicle) may be any material or molecular entity that facilitates the administration or other delivery of the pharmaceutical composition. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle

A therapeutically effective amount or concentration may be any amount of a composition that alone, or together with one or more additional therapeutic agents is sufficient to achieve a desired effect in a subject, or in a cell being treated with the agent. The effective amount of the agent will be dependent on several factors, including, but not limited to, the subject or cells being treated and the manner of administration of the therapeutic composition. In one example, a therapeutically effective amount or concentration is one that is sufficient to prevent advancement, delay progression, or to cause regression of a disease, or which is capable of reducing symptoms caused by any disease, including flavivirus infection. In one example, a desired response is to reduce or inhibit one or more symptoms associated with flavivirus infection. The one or more symptoms do not have to be completely eliminated for the composition to be effective. For example, a composition can decrease the sign or symptom by a desired amount, for example by at least 20%, at least 50%, at least 80%, at least 90%, at least 95%, at least 98%, or even at least 100%, as compared to the sign or symptom in the absence of the composition. A therapeutically effective amount of a disclosed pharmaceutical composition can be administered in a single dose, or in several doses, for example daily, during a course of treatment. However, the therapeutically effective amount can depend on the subject being treated, the severity and type of the condition being treated, and the manner of administration. For example, a therapeutically effective amount of such agent can vary from about 100 μg-10 mg per kg body weight if administered intravenously. The actual dosages of treatments for flavivirus infection will vary according to factors such as the type of flavivirus to be protected against (for example, DENV-1, DENV-2, DENV-3, DENV-4, WNV, or YFV) and the particular status of the subject (for example, the subject's age, size, fitness, extent of symptoms, susceptibility factors, and the like), time and route of administration, other drugs or treatments being administered concurrently, as well as the specific pharmacology of treatments for flavivirus infection for eliciting the desired activity or biological response in the subject. Dosage regimens can be adjusted to provide an optimum prophylactic or therapeutic response.

A therapeutically effective amount is also one in which any toxic or detrimental side effects of the compound and/or other biologically active agent is outweighed in clinical terms by therapeutically beneficial effects. A non-limiting range for a therapeutically effective amount of treatments for flavivirus infection within the methods and formulations of the disclosure is about 0.0001 μg/kg body weight to about 10 mg/kg body weight per dose, such as about 0.0001 μg/kg body weight to about 0.001 μg/kg body weight per dose, about 0.001 μg/kg body weight to about 0.01 μg/kg body weight per dose, about 0.01 μg/kg body weight to about 0.1 μg/kg body weight per dose, about 0.1 μg/kg body weight to about 10 μg/kg body weight per dose, about 1 μg/kg body weight to about 100 μg/kg body weight per dose, about 100 μg/kg body weight to about 500 μg/kg body weight per dose, about 500 μg/kg body weight per dose to about 1000 μg/kg body weight per dose, or about 1.0 mg/kg body weight to about 10 mg/kg body weight per dose.

Dosage can be varied by the attending clinician to maintain a desired concentration. Higher or lower concentrations can be selected based on the mode of delivery, for example, trans-epidermal, rectal, oral, pulmonary, intranasal delivery, intravenous or subcutaneous delivery.

Determination of effective dosages is typically based on animal model studies followed up by human clinical trials and is guided by administration protocols that significantly reduce the occurrence or severity of targeted disease symptoms or conditions in the subject. Suitable models in this regard include, for example, murine, rat, porcine, feline, non-human primate, and other accepted animal model subjects known in the art. Alternatively, effective dosages can be determined using in vitro models (for example, viral titer assays or cell culture infection assays). Using such models, only ordinary calculations and adjustments are required to determine an appropriate concentration and dose to administer a therapeutically effective amount of the treatments for flavivirus infection (for example, amounts that are effective to alleviate one or more symptoms of flavivirus infection).

### Vaccines

Administration of the disclosed compositions can provide an antigenic, immunological or protective (vaccine) response. More in particular, the immunization with the disclosed compositions can elicit antibodies.

The administration procedure for the disclosed antigenic compositions or viral vector that expresses said antigenic composition, compositions of the invention such as immunological, antigenic or vaccine compositions or therapeutic compositions can be via a parenteral route (intradermal, intramuscular, or subcutaneous). Such an administration enables a systemic immune response. The administration can be via a mucosal route, e.g., oral, nasal, genital, etc. Such an administration enables a local immune response.

More generally, the inventive antigenic, immunological or vaccine compositions or therapeutic compositions can be prepared in accordance with standard techniques well known to those skilled in the pharmaceutical arts. Such compositions can be administered in dosages and by techniques well known to those skilled in the medical arts taking into consideration such factors as the breed or species, age, sex, weight, and condition of the particular patient, and the route of administration. The compositions can be administered alone, or can be co-administered or sequentially administered with other compositions of the invention or with other immunological, antigenic or vaccine or therapeutic compositions. Such other compositions can include purified native antigens or epitopes or antigens or epitopes from the expression by an expression vector system; and are administered taking into account the aforementioned factors.

Examples of vaccine compositions include liquid preparations for orifice, e.g., oral, nasal, anal, genital, e.g., vaginal, etc., administration such as suspensions, syrups or elixirs; and, preparations for parenteral, subcutaneous, intradermal, intramuscular or intravenous administration (e.g., injectable administration) such as sterile suspensions or emulsions. In such compositions the recombinant may be in admixture with a suitable carrier, diluent, or excipient such as sterile water, physiological saline, glucose or the like.

Antigenic, immunological or vaccine compositions typically can contain an adjuvant and an amount of the disclosed compositions (or of a viral vector engineered to express the disclosed compositions) to elicit the desired response. In human applications, alum (aluminum phosphate or aluminum hydroxide) is a typical adjuvant. Saponin and its purified component Quil A, Freund's complete adjuvant and other adjuvants used in research and veterinary applications have toxicities which limit their potential use in human vaccines. Chemically defined preparations such as muramyl dipeptide, monophosphoryl lipid A, phospholipid conjugates such as those described by Goodman-Snitkoff et al. J. Immunol. 147:410-415 (1991) and incorporated by reference herein, encapsulation of the protein within a proteoliposome as described by Miller et al., J. Exp. Med. 176:1739-1744 (1992) and incorporated by reference herein, and encapsulation of the protein in lipid vesicles such as Novasome lipid vesicles (Micro Vescular Systems, Inc., Nashua, N.H.) can also be used.

The composition may be packaged in a single dosage form for immunization by parenteral (i.e., intramuscular, intradermal or subcutaneous) administration or orifice administration, e.g., perlingual (i.e., oral), intragastric, mucosal including intraoral, intraanal, intravaginal, and the like administration. And again, the effective dosage and route of administration are determined by the nature of the composition, by the nature of the expression product, by expression level if a viral expression vector is directly used, and by known factors, such as breed or species, age, sex, weight, condition and nature of host, as well as LD₅₀ and other screening procedures which are known and do not require undue experimentation. Dosages of expressed product can range from a few to a few hundred micrograms, e.g., 5 to 500 μg. The composition can be administered in any suitable amount to achieve expression at these dosage levels. A vaccine provided in a viral expression vector is administered in an amount of at least 10² pfu; thus, the inventive recombinant is can be administered in at least this amount; or in a range from about 10² pfu to about 10² pfu. Other suitable carriers or diluents can be water or a buffered saline, with or without a preservative. The expression product or viral expression vector can be lyophilized for resuspension at the time of administration or can be in solution.

The carrier can also be a polymeric delayed release system. Synthetic polymers are particularly useful in the formulation of a composition having controlled release. An early example of this was the polymerization of methyl methacrylate into spheres having diameters less than one micron to form so-called nano particles, reported by Kreuter, J., Microcapsules and Nanoparticles in Medicine and Pharmacology, M. Donbrow (Ed). CRC Press, p. 125-148.

Microencapsulation has been applied to the injection of microencapsulated pharmaceuticals to give a controlled release. A number of factors contribute to the selection of a particular polymer for microencapsulation. The reproducibility of polymer synthesis and the microencapsulation process, the cost of the microencapsulation materials and process, the toxicological profile, the requirements for variable release kinetics and the physicochemical compatibility of the polymer and the antigens are all factors that must be considered. Examples of useful polymers are polycarbonates, polyesters, polyurethanes, polyorthoesters and polyamides, particularly those that are biodegradable.

A frequent choice of a carrier for pharmaceuticals and more recently for antigens is poly (d,1-lactide-co-glycolide) (PLGA). This is a biodegradable polyester that has a long history of medical use in erodible sutures, bone plates and other temporary prostheses where it has not exhibited any toxicity. A wide variety of pharmaceuticals including peptides and antigens have been formulated into PLGA microcapsules. A body of data has accumulated on the adaption of PLGA for the controlled release of antigen, for example, as reviewed by Eldridge, J. H., et al. Current Topics in Microbiology and Immunology. 1989, 146:59-66. The entrapment of antigens in PLGA microspheres of 1 to 10 microns in diameter has been shown to have a remarkable adjuvant effect when administered orally. The PLGA microencapsulation process uses a phase separation of a water-in-oil emulsion. The compound of interest is prepared as an aqueous solution and the PLGA is dissolved in a suitable organic solvent such as methylene chloride and ethyl acetate. These two immiscible solutions are co-emulsified by high-speed stirring. A non-solvent for the polymer is then added, causing precipitation of the polymer around the aqueous droplets to form embryonic microcapsules. The microcapsules are collected, and stabilized with one of an assortment of agents (polyvinyl alcohol (PVA), gelatin, alginates, polyvinylpyrrolidone (PVP), or methylcellulose) and the solvent removed by either drying in vacuo or solvent extraction. Thus, solid, including solid-containing-liquid, liquid, and gel (including “gel caps”) compositions are envisioned.

Additionally, the disclosed antigens and viral expression vectors that express them can stimulate an immune or antibody response in animals. From those antibodies, by techniques well-known in the art, monoclonal antibodies can be prepared and, those monoclonal antibodies, can be employed in well-known antibody binding assays, diagnostic kits or tests to determine the presence or absence of antigen(s) and therefrom the presence or absence of the natural causative agent of the antigen or, to determine whether an immune response to that agent or to the antigen(s) has simply been stimulated.

The compositions of the invention may be injectable suspensions, solutions, sprays, lyophilized powders, syrups, elixirs and the like. Any suitable form of composition may be used. To prepare such a composition, a nucleic acid or vector of the invention, having the desired degree of purity, is mixed with one or more pharmaceutically acceptable carriers and/or excipients. The carriers and excipients must be “acceptable” in the sense of being compatible with the other ingredients of the composition. Acceptable carriers, excipients, or stabilizers are nontoxic to recipients at the dosages and concentrations employed, and include, but are not limited to, water, saline, phosphate buffered saline, dextrose, glycerol, ethanol, or combinations thereof, buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN® PLURONICS® or polyethylene glycol (PEG).

An immunogenic or immunological composition can also be formulated in the form of an oil-in-water emulsion. The oil-in-water emulsion can be based, for example, on light liquid paraffin oil (European Pharmacopea type); isoprenoid oil such as squalane, squalene, EICOSANE™ or tetratetracontane; oil resulting from the oligomerization of alkene(s), e.g., isobutene or decene; esters of acids or of alcohols containing a linear alkyl group, such as plant oils, ethyl oleate, propylene glycol di(caprylate/caprate), glyceryl tri(caprylate/caprate) or propylene glycol dioleate; esters of branched fatty acids or alcohols, e.g., isostearic acid esters. The oil advantageously is used in combination with emulsifiers to form the emulsion. The emulsifiers can be nonionic surfactants, such as esters of sorbitan, mannide (e.g., anhydromannitol oleate), glycerol, polyglycerol, propylene glycol, and oleic, isostearic, ricinoleic, or hydroxystearic acid, which are optionally ethoxylated, and polyoxypropylene-polyoxyethylene copolymer blocks, such as the Pluronic® products, e.g., L121. The adjuvant can be a mixture of emulsifier(s), micelle-forming agent, and oil such as that which is commercially available under the name Provax® (IDEC Pharmaceuticals, San Diego, Calif.).

The immunogenic compositions of the invention can contain additional substances, such as wetting or emulsifying agents, buffering agents, or adjuvants to enhance the effectiveness of the vaccines (Remington's Pharmaceutical Sciences, 18th edition, Mack Publishing Company, (ed.) 1980).

Adjuvants may also be included. Adjuvants include mineral salts (e.g., AlK(SO₄)₂, AlNa(SO₄)₂, AlNH(SO₄)₂, silica, alum, Al(OH)₃, Ca₃(PO₄)₂, kaolin, or carbon), polynucleotides with or without immune stimulating complexes (ISCOMs) (e.g., CpG oligonucleotides, such as those described in Chuang, T. H. et al, (2002) J. Leuk. Biol. 71(3): 538-44; Ahmad-Nejad, P. et al (2002) Eur. J. Immunol. 32(7): 1958-68; poly IC or poly AU acids, polyarginine with or without CpG (also known in the art as IC31; see Schellack, C. et al (2003) Proceedings of the 34th Annual Meeting of the German Society of Immunology; Lingnau, K. et al (2002) Vaccine 20(29-30): 3498-508), JuvaVax® (U.S. Pat. No. 6,693,086), certain natural substances (e.g., wax D from Mycobacterium tuberculosis, substances found in Cornyebacterium parvum, Bordetella pertussis, or members of the genus Brucella), flagellin (Toll-like receptor 5 ligand; see McSorley, S. J. et al (2002) J. Immunol. 169(7): 3914-9), saponins such as QS21, QS17, and QS7 (U.S. Pat. Nos. 5,057,540; 5,650,398; 6,524,584; 6,645,495), monophosphoryl lipid A, in particular, 3-de-O-acylated monophosphoryl lipid A (3D-MPL), imiquimod (also known in the art as IQM and commercially available as Aldara®; U.S. Pat. Nos. 4,689,338; 5,238,944; Zuber, A. K. et al (2004) 22(13-14): 1791-8), and the CCRS inhibitor CMPD167 (see Veazey, R. S. et al (2003) J. Exp. Med. 198: 1551-1562).

Aluminum hydroxide or phosphate(alum) are commonly used at 0.05 to 0.1% solution in phosphate buffered saline. Other adjuvants that can be used, especially with DNA vaccines, are cholera toxin, especially CTA1-DD/ISCOMs (see Mowat, A. M. et al (2001) J. Immunol. 167(6): 3398-405), polyphosphazenes (Allcock, H. R. (1998) App. Organometallic Chem. 12 (10-11): 659-666; Payne, L. G. et al (1995) Pharm. Biotechnol. 6: 473-93), cytokines such as, but not limited to, IL-2, IL-4, GM-CSF, IL-12, IL-15 IGF-1, IFN-α, IFN-β, and IFN-γ (Boyer et al., (2002) J. Liposome Res. 121:137-142; WO01/095919), immunoregulatory proteins such as CD40L (ADX40; see, for example, WO03/063899), and the CD1a ligand of natural killer cells (also known as CRONY or α-galactosyl ceramide; see Green, T. D. et al, (2003) J. Virol. 77(3): 2046-2055), immunostimulatory fusion proteins such as IL-2 fused to the Fc fragment of immunoglobulins (Barouch et al., Science 290:486-492, 2000) and co-stimulatory molecules B7.1 and B7.2 (Boyer), all of which can be administered either as proteins or in the form of DNA, in the same viral vectors as those encoding the antigens of the invention or on separate expression vectors. Alternatively, vaccines of the invention may be provided and administered without any adjuvants.

The immunogenic compositions can be designed to introduce the viral vectors to a desired site of action and release it at an appropriate and controllable rate. Methods of preparing controlled-release formulations are known in the art. For example, controlled release preparations can be produced by the use of polymers to complex or absorb the immunogen and/or immunogenic composition. A controlled-release formulation can be prepared using appropriate macromolecules (for example, polyesters, polyamino acids, polyvinyl, pyrrolidone, ethylenevinylacetate, methylcellulose, carboxymethylcellulose, or protamine sulfate) known to provide the desired controlled release characteristics or release profile. Another possible method to control the duration of action by a controlled-release preparation is to incorporate the active ingredients into particles of a polymeric material such as, for example, polyesters, polyamino acids, hydrogels, polylactic acid, polyglycolic acid, copolymers of these acids, or ethylene vinylacetate copolymers. Alternatively, instead of incorporating these active ingredients into polymeric particles, it is possible to entrap these materials into microcapsules prepared, for example, by coacervation techniques or by interfacial polymerization, for example, hydroxymethylcellulose or gelatin-microcapsule and poly-(methylmethacrylate) microcapsule, respectively, in colloidal drug delivery systems (for example, liposomes, albumin microspheres, microemulsions, nano-particles and nanocapsules) or in macroemulsions. Such techniques are disclosed in New Trends and Developments in Vaccines, Voller et al. (eds.), University Park Press, Baltimore, Md., 1978 and Remington's Pharmaceutical Sciences, 16th edition.

Suitable dosages of the viral vectors of the invention (collectively, the immunogens) in the immunogenic composition of the invention can be readily determined by those of skill in the art. For example, the dosage of the immunogens can vary depending on the route of administration and the size of the subject. Suitable doses can be determined by those of skill in the art, for example by measuring the immune response of a subject, such as a laboratory animal, using conventional immunological techniques, and adjusting the dosages as appropriate. Such techniques for measuring the immune response of the subject include but are not limited to, chromium release assays, tetramer binding assays, IFN-γ ELISPOT assays, IL-2 ELISPOT assays, intracellular cytokine assays, and other immunological detection assays, e.g., as detailed in the text “Antibodies: A Laboratory Manual” by Ed Harlow and David Lane.

The immunogenic compositions can be administered using any suitable delivery method including, but not limited to, intramuscular, intravenous, intradermal, mucosal, and topical delivery. Such techniques are well known to those of skill in the art. More specific examples of delivery methods are intramuscular injection, intradermal injection, and subcutaneous injection. However, delivery need not be limited to injection methods.

Immunization schedules (or regimens) are well known for animals (including humans) and can be readily determined for the particular subject and immunogenic composition. Hence, the immunogens can be administered one or more times to the subject. Preferably, there is a set time interval between separate administrations of the immunogenic composition. While this interval varies for every subject, typically it ranges from 10 days to several weeks, and is often 2, 4, 6 or 8 weeks. For humans, the interval is typically from 2 to 6 weeks. In a particularly advantageous embodiment of the present invention, the interval is longer, advantageously about 5 weeks 10 weeks, 12 weeks, 14 weeks, 16 weeks, 18 weeks, 20 weeks, 22 weeks, 24 weeks, 26 weeks, 28 weeks, 30 weeks, 32 weeks, 34 weeks, 36 weeks, 38 weeks, 40 weeks, 42 weeks, 44 weeks, 46 weeks, 48 weeks, 50 weeks, 52 weeks, 54 weeks, 56 weeks, 58 weeks, 60 weeks, 62 weeks, 64 weeks, 66 weeks, 68 weeks or 70 weeks. The interval need not be an exact number of weeks. For example, an immunization performed after a five week interval, where the first immunization was performed on a Tuesday, need not also be performed on a Tuesday to result in a five week interval. It can be performed any number of days following the five week interval provided that a sixth week has not passed.

The immunization regimes typically have from 1 to 6 administrations of the immunogenic composition, but may have as few as one or two or four. The methods of inducing an immune response can also include administration of an adjuvant with the immunogens. In some instances, annual, biannual or other long interval (5-10 years) booster immunization can supplement the initial immunization protocol.

The present methods also include a variety of prime-boost regimens, for example DNA prime-Adenovirus boost regimens. In these methods, one or more priming immunizations are followed by one or more boosting immunizations. The actual immunogenic composition can be the same or different for each immunization and the type of immunogenic composition (e.g., containing protein or expression vector), the route, and formulation of the immunogens can also be varied. For example, if an expression vector is used for the priming and boosting steps, it can either be of the same or different type (e.g., DNA or bacterial or viral expression vector). One useful prime-boost regimen provides for two priming immunizations, four weeks apart, followed by two boosting immunizations at 4 and 8 weeks after the last priming immunization. It should also be readily apparent to one of skill in the art that there are several permutations and combinations that are encompassed using the DNA, bacterial and viral expression vectors of the invention to provide priming and boosting regimens.

The immunogenic compositions of the invention can be administered alone, or can be co-administered, or sequentially administered, with other antigens, e.g., with “other” immunological, antigenic or vaccine or therapeutic compositions thereby providing multivalent or “cocktail” or combination compositions of the invention and methods of employing them. Again, the ingredients and manner (sequential or co-administration) of administration, as well as dosages can be determined taking into consideration such factors as the age, sex, weight, species and condition of the particular subject, and the route of administration.

Although the present invention and its advantages have been described in detail, it should be understood that various changes, substitutions and alterations can be made herein without departing from the spirit and scope of the invention as defined in the appended claims.

### EXAMPLES

The following examples are for illustration only. In light of this disclosure, those of skill in the art will recognize that variations of these examples and other examples of the disclosed invention be possible without undue experimentation.

### Example 1—Animals and Immunizations

Adult Macaca mulatta (rhesus macaques) were housed at the Oregon National Primate Research Center (ONPRC) in Beaverton, Oreg. All procedures were performed according to protocols approved by the IACUC at OHSU. A total of six macaques were co-immunized with DENV E domain III gene expression plasmids and soluble EDIII-E2 particles at weeks 0, 4, and 12. At each immunization a total of 36 μg DNA was delivered epidermally by Particle Mediated Epidermal Delivery (PMED) device (gene gun, XR-1 research model, PowderMed, Oxford, UK), by administering 1.8 μg of the DNA vaccines with 0.2 μg of Rhesus IL-12 adjuvant plasmid (2 μg total) coated onto 1 μg of 1 μM-size gold particles into each of 18 sites along the shaved abdomen and upper thighs. Simultaneously, 500 μg of soluble EDIII-E2 particles was delivered intramuscularly formulated with 20% Adjuplex (Sigma) adjuvant. Regular blood draws monitored immune responses in sera.

### Example 2—DNA Vaccine Gene Gun Cartridges

DNA was precipitated onto 1 μm diameter gold beads, and cartridges were prepared for delivery with the PowderMed PMED gene gun delivery device as described according to the manufacturer's instructions (Bio-Rad, Hercules, Calif.).

### Example 3—Recombinant EDIII-E2 Particles

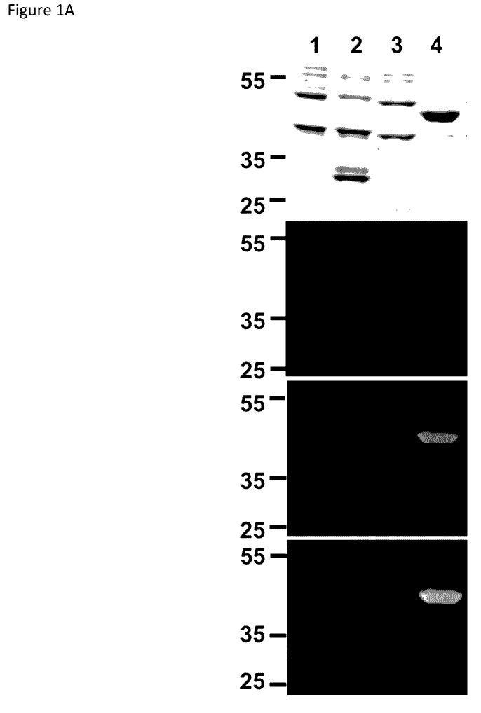

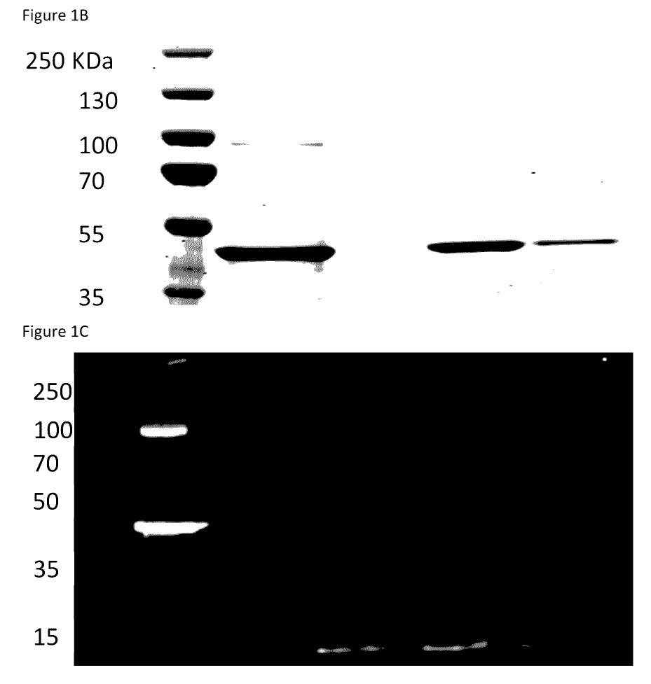

The EDIII-E2 expression vectors were constructed from the previously described pETE2DISP plasmid [21]. The oligonucleotide sequence encoding the serotype 2 DENV isolate 16681 E domain III (amino acid (aa) 297-399, positions on the DENV-2 E glycoprotein) was cloned into the pETE2DISP vector for expression of EDIII as an N-terminal fusion to the E2 core scaffold (FIG. 1A). The oligonucleotide sequence encoding EDIII was amplified from strain 16681 clone plasmid using primers (D2-ED3+) NNNNCCATGCCGATGTCATACTCTATGTGCACAGG (SEQ ID NO: 3) and (D2-ED3-) NNNNCCCGGGGCCGATAGAACTTCCTTTCTTAAAC (SEQ ID NO: 4) containing the restriction site Xmal. Cycling conditions for the PCR were as follows: denature at 94° C. for 2 min, 10× (94° C. for 15 sec, 49° C. for 30 sec, and 72° C. for 60 sec), 30× (94° C. for 15 sec, 65° C. for 30 sec, and 72° C. for 60 sec), and a final elongation of 72° C. for 7 min. The PCR product and the pETE2DISP vector were digested with Nhel (New England Biolabs) and ligated together with T4 DNA ligase (New England Biolabs) before transformation into BL21 (DE3) CodonPlus-RIPL competent cells (Stratagene). In-frame ligation was confirmed by sequencing. The construct encoding N-terminal fusions of EDIII to the E2 scaffold are annotated in this document as EDIII-E2.

### Example 4—Expression, Purification and Refolding of Env-E2 Multimeric Scaffolds

The plasmid encoding EDIII-E2 fusion protein was maintained and expressed in BL21 (DE3) CodonPlus-RIPL cells (Stratagene). Cells were grown overnight at 37° C. in Luria-Bertani (LB) broth with a final concentration of 100 μg/ml ampicillin and 50 μg/ml of chloramphenicol, shaking at 225 rpm. The following day, the cultures were back diluted 1:20 and grown to an OD₆₀₀ between 0.8-1.0. Protein expression was induced with the addition of Isopropyl β-D-1 thiogalactopyranoside (IPTG) with a final concentration of 1 mM. Cells were harvested by centrifugation at 5000 g for 5 min at 4° C. Pellets were then resuspended in Lysis buffer (lx BugBuster Protein Extraction Reagent (Novagen), 1×PBS, Benzonase Nuclease (EMD), rLysozyme (EMD), TurboDNase (Ambion), Cyanase (RiboSolutions) and Complete, EDTA-free Protease Inhibitor Cocktail Tablet (Roche)) and incubated at room temperature for 30 min and then at 37° C. for 30 min, shaking at 225 rpm.

The soluble fraction containing the E2 monomers was recovered after centrifugation at 10,000 g for 10 min at 4° C. and was loaded onto a Sephadex G-25 column (GE Healthcare) for buffer exchange. Fractions containing E2 wt were pooled and loaded onto a Detoxi-gel column (Pierce). E2 wt did not bind to the Detoxi-gel column and was recovered in the flow through, which was loaded onto a Q-Sepharose anion exchange column (GE Healthcare) at 1.0 ml/min. Bound protein was eluted from the column with a 0-60%/400 ml gradient of elution buffer (20 mM Tris-HCl, pH 8.5, 1 mM EDTA, 1M NaCl) at 3.0 ml/min., with the E2 wt eluting at a salt concentration of 30%. Peak fractions containing E2 wt were pooled and concentrated with a 10 kD molecular weight cut off (MWCO) using Amicon Ultra Centrifugal Filter (Millipore). The retentate was loaded onto a Superdex200 gel filtration column (GE Healthcare) at 1 ml/min using Solubility Buffer 2.2 (1×PBS, 50 mM L-Glutamine (Sigma), 50 mM NaCl, 250 mM L-Arginine (Sigma)). Fractions containing the 1.5 MDa E2 wt 60-mer particles were concentrated to 1 mg/ml using the Ultra Centrifugal devices and then stored in Solubility Buffer 2.2 at −80° C.

The EDIII-E2 fusion proteins formed inclusion bodies in E. coli and were purified from the insoluble fraction following bacterial lysis and centrifugation at 10,000 g for 10 min at 4° C. Inclusion bodies were washed three times with Inclusion Body Wash Buffer (1M Guanidine Hydrochloride (GuHCl), 50 mM NaCl, 1 mM DTT, 1×PBS, 10% glycerol, 0.5 M Arginine, pH 7.4) before dissolving in Unfolding Buffer (6M GuHCl, 1 mM DTT, 1×PBS). EDIII-E2 inclusion bodies were allowed to unfold in Unfolding Buffer (6M GuHCl, 1 mM DTT, 1×PBS) rocking at 4° C. for a minimum of 3 h. The proteins were transferred to SnakeSkin dialysis tubing, 10K MWCO (Pierce) and subjected to step-down dialysis against the following buffers: 4M GuHCl (4M GuHCl, 50 mM NaCl, 1 mM DTT, 1×PBS, 10% glycerol, 0.5 M Arginine, pH 8.0), 2M GuHCl (2M GuHCl, 50 mM NaCl, 0.5 mM DTT, 1×PBS, 10% glycerol, 0.5M Arginine, pH 8.0), 0M GuHCl#1 (50 mM NaCl, 1×PBS, 10% glycerol, 0.5M Arginine, 0.5 mM reduced glutathione, 0.1 mM oxidized glutathione, pH 8.0), 0M GuHCl#2 (50 mM NaCl, 1×PBS, 10% glycerol, 0.5M Arginine, 0.1 mM reduced glutathione, 0.1 mM oxidized glutathione, pH 8.0), and 0M GuHCl#3 (50 mM NaCl, 1×PBS, 10% glycerol, 0.5M Arginine, pH 8.0). A final dialysis was performed in Solubility Buffer 2.2. Refolded soluble 60mer particles were confirmed by gel filtration using the Superdex200 gel filtration column (GE Healthcare), and purity and identity were assessed by SDS-PAGE and Western blot analysis, respectively. Purified multimeric proteins were stored at −80° C.

### Example 5—SDS-PAGE and Western Blot Analysis

Expression, refolding, and identity of recombinant proteins were assessed by SDS-PAGE and Western blot analysis using Invitrogen NuPAGE 4-12% Bis-Tris mini-gels (Carlsbad, Calif.) under reducing conditions. For SDS-PAGE, gels were stained with SimplyBlue SafeStain (Invitrogen). For Western blot analysis, proteins were transferred onto nitrocellulose paper (Invitrogen), blocked with Odyssey blocking buffer (LI-COR Biosciences) overnight at 4° C. The following day, the blot was probed simultaneously with serum from a rabbit immunized with E2 wt (1:8000) and the mouse mAbs 8A5 (1:2,000) for 1 h at room temperature. Primary Abs were prepared in Odyssey Blocking Buffer 1:1 with 1×PBS, 0.2% Tween-20. Blots were washed 5 times with 0.1% Triton X-100, 1×PBS. Secondary Abs IRDye 680 Goat anti-Rabbit and IRDye 800CW Goat anti-mouse (LI-COR Biosciences) were used at 1:15,000, diluted in Odyssey Blocking Buffer 1:1 with 1×PBS, 0.2% Tween-20, 0.02% SDS. Membranes were scanned using the LI-COR Odyssey Infrared Imaging System (LI-COR Biosciences) to allow simultaneous two-color detection of E2 and the DENV EDIII. Integrated intensities were used in conjunction with protein concentrations determined by NanoDrop (NanoDrop Technologies, Wilmington Del.) to calculate protein purity and concentration.

### Example 6—Biosensor Analyses

Surface plasmon resonance (SPR) biosensor assays were carried out at 25° C. using the Biacore T200 instrument (GE Healthcare, Piscataway, N.J.). For all experiments, 10 μg/ml of E2-wt or EDIII-E2 particles were diluted in sodium acetate buffer (pH 5.5) and immobilized by standard amine coupling chemistry on a CM3 sensor chip. E2 wt and EDIII-E2 particles were immobilized to a level of 60 RU on flow cell 2 and 3, respectfully. Flow cell 1 was activated and blocked and its response was subtracted from all other flow cells. Binding experiments were carried out by injecting the monoclonal Abs 8A5 over the sensor surface at concentrations of 0 nM, 11.1 nM, 33.3 nM(2), 100 nM and 300 nM in HBS-EP buffer (10 mM HEPES, 150 mM NaCl, 1 mM EDTA, 0.05% P20) at a flow rate of 30 μI/min for 5 minutes (association phase). Following a 10-minute dissociation phase, the chip surface was regenerated for each concentration of injected Ab with a 45 second pulse of 10 mM glycine, pH 2.5. Additionally mAb DVC 3.7, DVC 10.16 and DVC 14.2 were tested at a single concentration of 33.3 nM. All data were double reference subtracted with buffer blank injections. Data were processed using T200 evaluation software.

### Example 7—Enzyme-Linked Immunosorbent Assay (ELISA)

Binding Ab responses from individual macaques to the E2 wt protein and EDIII were measured by ELISA as described previously [38]. Endpoint titers were calculated as the lowest positive value for each sample that was three times the average background of pre-immune macaque serum included in triplicate on each plate. Individual peptides were added (0.05 ml at 0.01 mg/ml) to flat-bottom 96-well plates (Thermo Scientific), and then the plates were incubated overnight at 4° C. Plates were blocked with Blotto (PBS, 5% nonfat dry milk, 1% goat serum) for 1 hour. Serum was then incubated for 1 hour at room temperature, and then washed five times with Wash Buffer (PBS, 0.1% Triton X-100). Goat anti-human IgG-HRP at 1:4000 in Disruption Buffer (PBS, 5% FBS, 2% BSA, 1% Triton X-100) was then added to the plate and incubated for one hour at room temperature. The plates were washed, and TMB substrate (Sigma) was added to each well and incubated for 30 minutes in the dark. The reaction was stopped with 1N H₂SO₄, and the plates were read at 450 nm (Molecular Devices SpectraMax 190). Optical density (OD) values were calculated for each NHP.

### Example 8—Production of Dengue Viral Stocks

DENV-2 strains 16681 and 16803 were used in all experiments. Strain 16681 was derived from the previously described DENV-2 16681 infectious clone (Kinney R M et al, Virology 230, 300-308 (1997); incorporated by reference herein). Viruses were propagated in Aedes albopictus C6/36 cells adapted to grow at 32° C. in MEM (Cellgro) supplemented with 5% fetal bovine serum (FBS), Non-essential amino acids (Cellgro) and antibacterial-antifungal mix (Gibco anti-anti). Virus was applied to 80-90% confluent monolayers at an approximate multiplicity of infection (MOI) of 0.01 and incubated for 5-7 days. Culture supernatants were harvested, clarified by centrifugation and frozen at −80° C. in 10% sucrose-phosphate-glutamic acid (SPG) buffer.

### Example 9—Focus Assays

The focus is based on a method previously described in Whitehead S S et al, J Virol 77, 1653-1657 (2003); incorporated by reference herein. Briefly, twenty-four well plates were seeded with 5×10⁴ Vero cells in MEM supplemented with 5% fetal bovine serum (FBS) and grown for 24 hours. Growth media was removed. For virus titration, virus stocks were diluted serially ten-fold and added to individual wells. Cells were overlaid with 1 ml 0.8% methylcellulose in OptiMEM (Gibco) supplemented with 2% FBS (Cellgro) and antibiotic mix (Gibco Anti-Anti) and incubated 5 days at 37° C., 5% CO₂. On day 5, overlay was removed, cells washed with PBS, fixed in 80% methanol and developed. To develop plates, fixed monolayers were blocked with 5% instant milk PBS, followed by incubation with anti-flavivirus MAb 4G2 diluted 1:1000. Wells were washed with PBS and incubated with horseradish peroxidase (HRP) conjugated goat anti-mouse Ab (Sigma) diluted 1:500 in blocking buffer for 1 hr at 37° C. Plates were washed once in PBS and foci developed by the addition of 100 ul of TrueBlue HRP substrate (KPL). Foci were counted on a light box and viral titers calculated by standard methods. For focus reduction neutralization test (FRNT), primate sera were serially diluted four-fold from starting dilutions of 1:10 or 1:20. Each dilution was mixed with approximately 50 focus forming units (ffu) of virus to a final volume of 200 μl, incubated for 1 hour at 37° C., 5% CO₂ and added in triplicate to 24 wells plates and processed as above. For delayed focus assay, 150 uL of acute MACAQUE sera were inoculated onto 90% confluent C6/36 cells in T-25 flasks, incubated for one hour, overlaid with MEM supplemented with 5% FBS and incubated for 7 days at 32° C., 5% CO₂. On day 7, supernatants were harvested and titrated as described above.

### Example 10—Dengue Virus Challenge

The six vaccinated macaques (EDIII-E2) and three additional naïve macaques were challenged with serotype 2 viral DENV isolate 16803. The viral challenge was conducted five weeks following the final vaccination. Animals were challenged with 5×10⁵ FFU in 0.2 mL delivered by intramuscular injection in the quadriceps muscle. Serum samples were collected at the time of viral challenge as well as daily for the first ten days post-challenge. A final blood draw was also conducted at day 21 post-infection.

### Example 11—RNA Isolation and Quantification

Blood specimens collected on days 0 through 10 were collected and assayed for viral RNA by routine and quantitative PCR. QiaAmp Viral RNA mini Kit (QIAGEN, Valencia, Calif.) was used to extract viral RNA from primate sera following the manufacturer's protocol. Extracted RNA was immediately subjected to routine RT-PCR as previously described (Lanciotti R S et al, J Clin Microbiol 30, 545-551 (1992); incorporated by reference herein). RNA from PCR positive serum samples were subsequently subjected to single reaction real-time RT-PCR as previously described (Waggoner J J et al, J Clin Microbiol 51, 3418-3420 (2013); incorporated by reference herein) using an Applied Biosystems Step-One Plus thermocycler (Forest City, Calif.).

### Example 12—Recombinant EDIII-E2 Fusion Protein Preserves EDIII Conformational Epitopes

A nucleic acid fragment encoding the DIII region (SEQ ID NO: 1) was amplified using PCR from DENV-2 16681 infectious clone plasmid [26] using oligonucleotide primers SEQ ID NO: 3 and SEQ ID NO: 4 and then ligated in frame to the 5′ end of the E2 gene within the pE2IDSP expression vector, generating an EDIII-E2 fusion gene. The resulting EDIII-E2 fusion protein was expressed in E. coli and purified from inclusion bodies. Cell lysates were obtained and protein expression analyzed by SDS-PAGE (FIG. 1A). The post-induction samples contained the ^{˜}50 Kda predicted band from the EDIII-E2 fusion protein (FIG. 1A). These samples were further characterized by western blot using both E2 specific and EDIII specific antibodies (FIG. 1A). The cell lysates contained both the E2 and EDIII proteins that co-localized in a single band. Solubilized fusion proteins were refolded using step-down dialysis. The final refolded proteins were purified by gel filtration. The purified vaccine construct was evaluated by SDS-PAGE and results indicate that the resulting protein is greater than 95% pure (FIG. 1B).

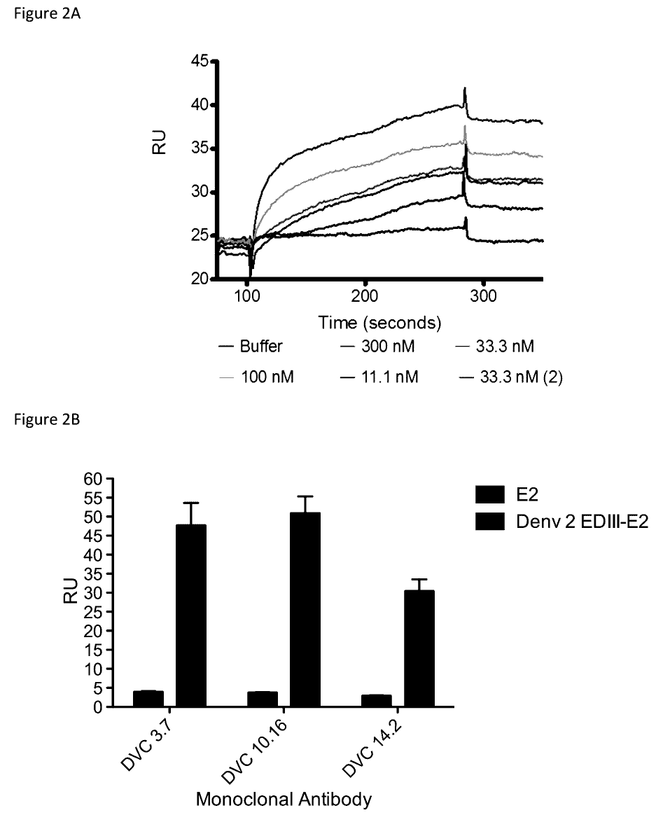

Matching the EDIII-E2 region, the DIII region (SEQ ID NO: 1) was amplified as described earlier from serotype 2 DENV 16681 infectious clone plasmid and ligated into a DNA expression vector, generating an EDIII expressing plasmid. The vaccine plasmid was combined with an adjuvant plasmid expressing Rhesus IL-12 to generate the complete DNA component of the vaccine. Expression of the EDIII insert was verified by western blot (FIG. 1C). The preservation of native EDIII epitopes on the surface of the EDIII-E2 particle was determined by surface plasmon resonance (SPR). The conformation-dependent mouse monoclonal antibody (mAb) 8A5 was tested at multiple concentrations against E2 only particles as well as EDIII-E2 particles (FIG. 2A). Specific binding of 8A5 was only observed with the EDIII-E2 particles. Three additional human conformational EDIII specific mAbs (DVC 3.7, DVC 10.16 and DVC 14.21 (de Alwis R et al, PLoS Negl Trop Dis 5, e1188 (2011); incorporated by reference herein) were also tested against the E2 and EDIII-E2 particles. Again in each case specific antibody binding was only observed with the EDIII-E2 particle (FIG. 2B). These results indicate that EDIII on the surface of the EDIII-E2 particle is accessible and displayed in a native conformation.

### Example 13—the EDIII-E2 Vaccine is Highly Immunogenic

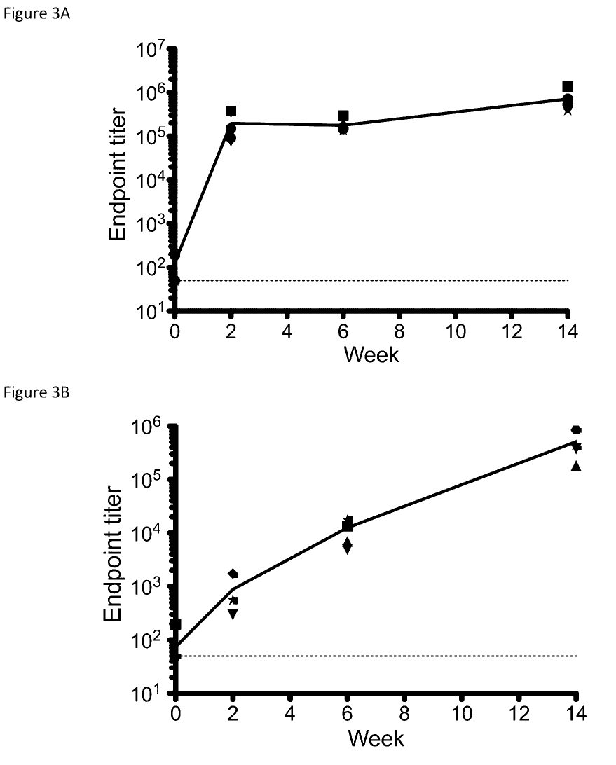

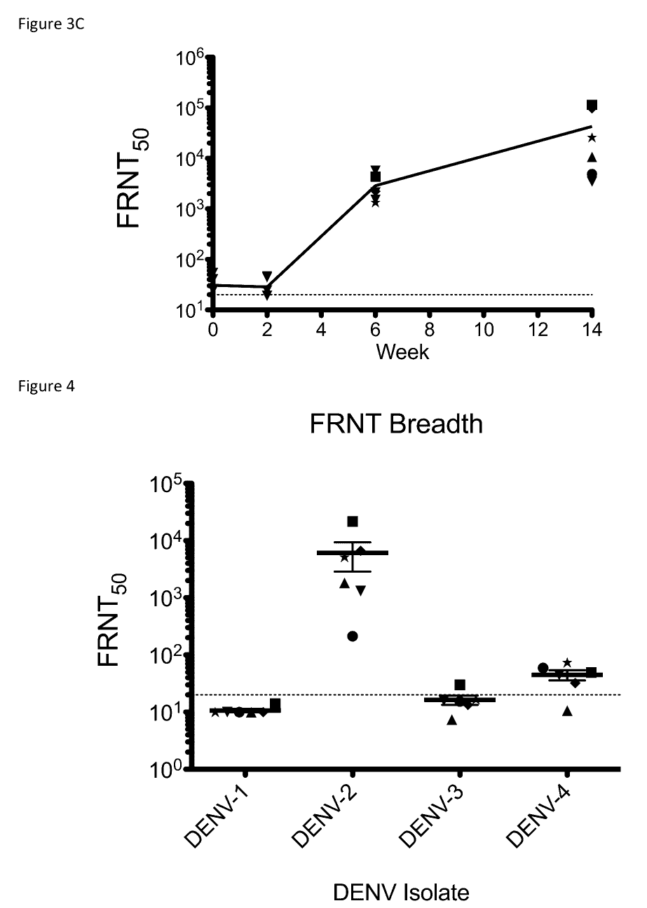

The full vaccine regimen consisted of both the EDIII-E2 purified particle delivered intramuscularly and the EDIII DNA expression plasmid delivered intradermally. The DNA vaccine contained also included a plasmid expressing rhesus IL-12 as an adjuvant. Expression of the DIII insert in 293T cells was verified by western blot (FIG. 1C). Adult macaques were inoculated with EDIII-E2 recombinant protein and EDIII DNA plasmid plus the rhesus IL-12 plasmid at weeks 0, 4, and 12. Blood was drawn prior to the initial inoculation as well as two weeks following each inoculation. Humoral responses to the E2 particle and EDIII were first evaluated by ELISA (FIGS. 3A and 3B). Strong and rapid antibody responses to the E2 carrier particle were observed following the initial vaccination with endpoint antibody titers reaching 1:100,000 (FIG. 3A) and was not significantly boosted by additional inoculations. Anti-EDIII binding antibody response was also detected following the first inoculation (FIG. 3B). These EDIII specific responses were boosted by each subsequent vaccination reaching a final endpoint titer of 1:100,000, similar to the maximum titer of the E2 responses, by the third dose. The development of neutralizing antibodies was evaluated by FRNT₅₀ to homologous D2 16681. FRNT₅₀ titers against DENV-2>1:20 were detected after the second vaccination (FIG. 3C) and were further boosted following the third vaccination. The breadth of the neutralizing antibody responses was measured by FRNT against heterologous DENV-1 (West Pac '74), DENV-3 (UNC3001ic), and DENV-4 (TVP360) viral isolates. Low levels of neutralization against DENV-4 isolate TVP360 was detected with a geometric mean titer (GMT) of 1:40, and no neutralization (<1:20 for all macaques) was detected against DENV-1 or DENV-3 (FIG. 4). Overall the EDIII-E2/DNA EDIII vaccine elicits a narrow, highly serotype specific neutralizing antibody response.

### Example 14—the EDIII-E2 Vaccine is Protective Against DENV-2 Challenge

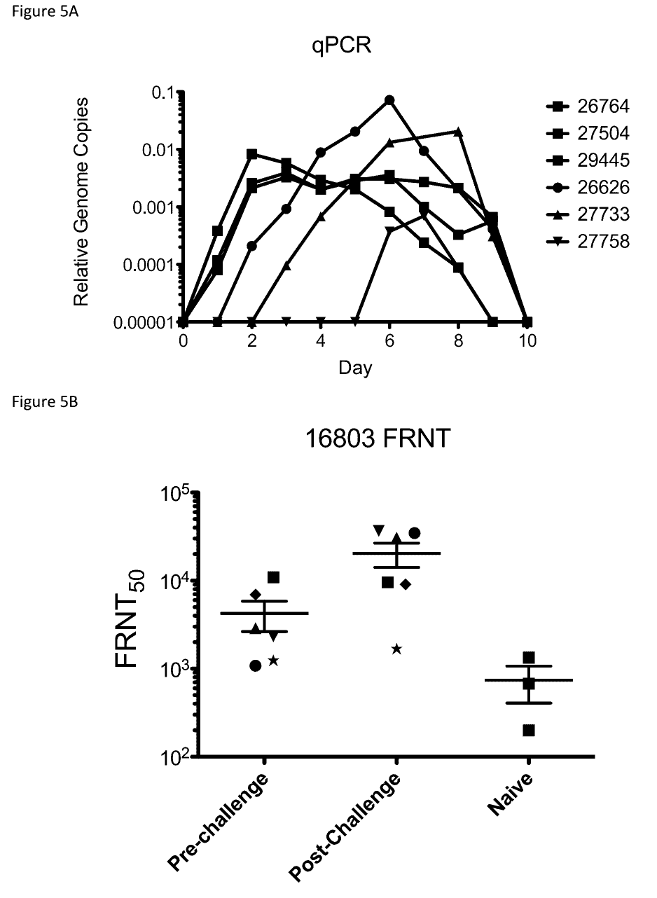

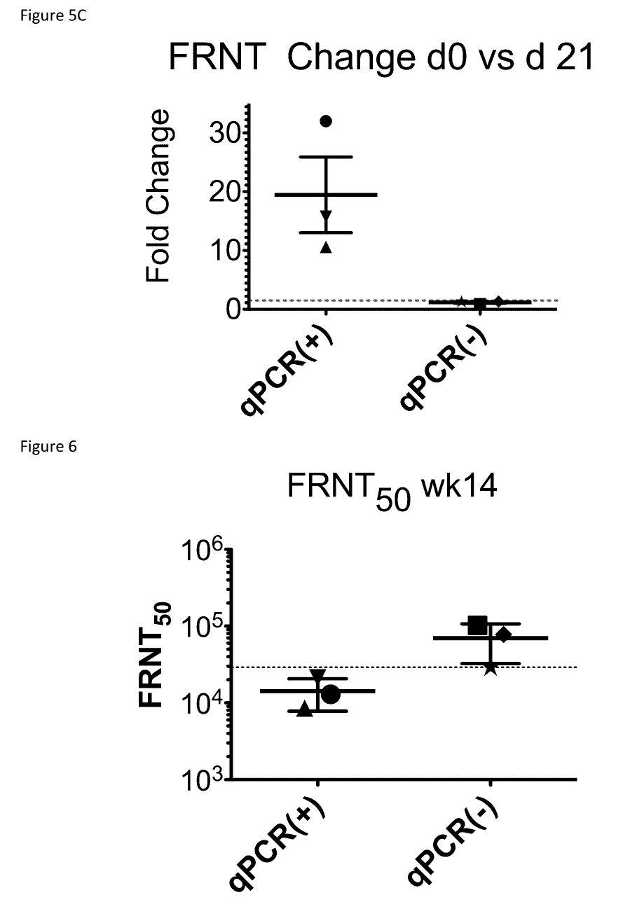

Vaccinated and naïve macaques were challenged with heterologous DENV-2 strain 16803 5 weeks following the final vaccination. Serum samples were obtained daily from day 0-10 and at 21 days post-challenge to verify viral clearance and to measure the early post-challenge immune responses. Systemic infectious viremia was determined by delayed focus assay (Table 1). All naïve macaques had detectable infectious virus by day two post-challenge with a duration range of 2-7 days. None of the vaccinated group had detectable infectious virus at any point post-challenge. The presence of viral RNA was determined by qPCR (FIG. 5A). As expected, all of the naïve controls had detectible dengue RNA levels over the 10 day course of the challenge. DENV-2 RNA was detected in 3 of 6 vaccinated macaques, with onset and duration of viral RNA trending towards being later and shorter than that observed for naïve animals. Infectious virus was not recovered from any of these macaques. The remaining 3 vaccinees were completely protected from DENV-2 infection.

FRNT₅₀ titers against the DENV-2 16803 were measured at d0 and d21 post-infection in the control group and d21 post-infection in the naïve group. All macaques in the vaccine group had FRNT₅₀ titers of greater that 1:1,000 at the time of challenge (FIG. 5B). At d21 post-challenge, the GMT FRNT₅₀ titer for the vaccine group increased more than 10 fold. However, the increase in FRNT₅₀ titer was only observed in those 3 animals that had detectable viral RNA levels (FIG. 5A), with a 10-30 fold boost in titer (FIG. 5C). FRNT₅₀ titers for the other 3 macaques remained constant, indicating that the EDIII-E2 vaccine had induced sterilizing immunity in these three animals (FIG. 5D). A week 14 antibody titer >1:20,000 against 16681 in the vaccinated macaques was associated with sterilizing immunity that provided absolute protection against challenge with 16803 (FIG. 6) and week 14 antibody titers were significantly negatively associated with days viremic in the vaccinated macaques (Spearman r=−0.83, P=0.033).

### Example 15—an EDIII-E2 Vaccine

The need for a safe and efficacious dengue vaccine is substantial. Several promising candidates are under development, the most advanced of which is the CYD-TDV vaccine, a LAV approach. However, recent clinical trial data have revealed that this approach has significant shortcomings, including a dependence on pre-existing seropositivity for maximum efficacy in vaccine recipients, a trend towards severe disease in hospitalized patients aged <9 yrs old, and highly variable immunogenicity against each serotype. While CYD-TDV induced detectable (FRNT₅₀>1:10) neutralizing antibodies in virtually all vaccine recipients tested, the results of the phase 3 trials clearly demonstrate that neutralizing antibodies detected at a minimum FRNT₅₀ of 1:10 against all four serotypes do not necessarily correlate with protection on challenge. In fact, taken together, the results of the CYD-TDV trials strongly suggest that FRNT₅₀ titers that correlate with protection following vaccination remain to be established. Limited immunogenicity because of live-attenuated viral interference and immunodominance (or inferiority) of individual serotypes is hypothesized to play a central role in the variable results of the CYD-TDV trials (Halstead S B, Vaccine 31, 4501-4507 (2013) and Halstead S B, Lancet 380, 1535-1536 (2012); incorporated by reference herein, and other LAV based approaches are expected to face similar hurdles. Non-LAV vaccines offer an alternate strategy that may have the potential to overcome these LAV challenges.

It is disclosed herein that recombinant DENV-2 EDIII displayed on a protein scaffold preserves native DENV-2 epitopes, is highly immunogenic in macaques when delivered with DNA expressing the same region and rhesus IL-12, and elicited protective immunity against DENV-2 challenge. Previous studies of EDIII based vaccines in RMs have variously reported “protection” from challenge by infectious assay or quantitative PCR (qPCR) and/or post challenge hemagglutination inhibition (HAI) or FRNT₅₀ titer boosting. HAI or neutralization titer boosting is an anamnestic response to challenge, and is considered indicative of at least low level viral replication. No non-human primate vaccine studies have previously reported all three readouts, even though all are critical to understanding the level of protection conferred by the vaccine, especially in the non-human primate model in which only viremia and not disease is measurable. Additionally, employing multiple complementary measures of protection can provide a more detailed and informative interpretation of necessary antibody thresholds of protection (Sariol C A and White L I, Front Immunol 4, 452 (2014); incorporated by reference herein). Using these three readouts, two distinct outcomes were observed in this study. First, evidence for sterilizing immunity was found in three vaccinated macaques, as demonstrated by the challenge virus being undetectable by infectious assay and quantitative PCR, as well as no evidence for a post challenge neutralization titers boost. Second, in the remaining three vaccinated macaques, evidence for partial protection was found. While challenge virus was not detected via infectious assay, but viral RNA by was detected by qPCR. In addition, a neutralization titer boost was observed 21 days post challenge, indicating low level viral replication after challenge. These results highlight the importance of using all three post-challenge methodologies in order to comprehensively characterize vaccine-induced protective immunity. Because NHP models to date do not result in disease readouts, viremia and anamnestic immune responses can be used to determine levels of infection but not disease.

A significant correlation was observed between week 14 FRNT₅₀ and viremia on challenge. Week 14 FRNT₅₀ titer was also predictive of sterile versus anamnestic immunity: the three macaques with FRNT₅₀ titers>1:25,000 were sterilely protected (GMT=61,435) whereas the three with FRNT₅₀ titers<1:25,000 had detectable viral RNA by qPCR (GMT=13,273 (FIG. 6) and a boost in 21 day post-challenge FRNT₅₀ titer (FIG. 5C). This result suggests that the threshold of protection, as defined by FRNT₅₀ against 16681 is around 1:25,000 (FIG. 6). The FRNT₅₀ titers associated with sterilizing immunity are considerably higher than what have been observed as a sterilizing protective neutralization threshold for humans vaccinated with LAV DENV vaccines (Sun W et al, J Infect Dis 207, 700-708 (2013); incorporated by reference herein), following natural infection (Buddhari D et al, PLoS Negl Trop Dis 8, e3230 (2014); incorporated by reference herein) and other vaccinated NHPs. The finding that the protective titer of neutralizing antibodies measured in vitro differ between recombinant vaccination and natural infection suggests that neutralization by antibodies raised against recombinant vaccine protein differs from antibodies raised by natural infection or LAV vaccination. Higher neutralizing antibody titers may have to be achieved by recombinant protein vaccines in macaques to establish sterilizing protection.

While FRNT₅₀ titer against the vaccine strain was predictive of sterilizing immunity, it is to be noted that FRNT₅₀ titers against the challenge strain were not. Against challenge strain 16803, two of the three macaques with sterile immunity (27350 and MACAQUE 2772) had FRNT₅₀ titers>1:6000 against 16803, the third macaque (27923) had a titer of only (1:1200), more than four-fold lower than against 16681. Despite this lower titer against the challenge strain, macaque 2793 was fully protected against challenge, as predicted by 16681 titer. This discrepancy is intriguing as 16681 and 16803 differ in only one residue on EDIII-E383D, This residue lies on the EDIII lateral ridge, an epitope region that is targeted by potently DENV neutralizing monoclonal antibodies (MAbs) (Sukupolvi-Petty S et al, J Virol 84, 9227-9239 (2010) and Sukupolvi-Petty S et al, J Virol 81, 12816-12826 (2007); both of which are incorporated by reference herein). Some of these MAbs have been shown to be sensitive to mutations near this position (Brien J D et al, J Virol 84, 10630-10643 (2010) and Zhou Y et al, Virology 439, 57-64 (2013); both of which are incorporated by reference herein. Future studies using recombinant DENV-2 clones should be developed to further explore this possibility and to more precisely dissect the polyclonal NAb response elicited by the E2-EDIII vaccine.

The E2 platform disclosed herein has the potential to be fully developed into a tetravalent DENV vaccine. Because the E2 protein stably displayed DENV-2 EDIII in a native conformation, the remaining serotype EDllls are likely to be similarly preserved. Intriguingly, multimeric E2 VLP can be assembled either using a cocktail of E2 monomers displaying each of the four EDIIIs or a refolded multi-serotype vaccine containing VLPs assembled from each individual serotype. Also disclosed is the approximate threshold antibody required for sterilizing immunity against DENV-2 in macaques, allowing vaccine evaluation to proceed with a clear endpoint. Further development of the EDIII-E2 DNA and particle vaccine approach will be focused on consistently inducing persistent neutralizing antibody titers above the threshold of protection disclosed herein.

TABLE 1
DENV 16803 Challenge-Detectable
Viremia by delayed focus assay.

| | | Day Post Infection | | | | | | | | | |
|---|---|---|---|---|---|---|---|---|---|---|---|
| Group | NHP | 1 | 2 | 3 | 4 | 5 | 6 | 7 | 8 | 9 | 10 |
| EDIII-E2 | 26626 | − | − | − | − | − | − | − | − | − | − |
| | 27350 | − | − | − | − | − | − | − | − | − | − |
| | 27733 | − | − | − | − | − | − | − | − | − | − |
| | 27758 | − | − | − | − | − | − | − | − | − | − |
| | 27772 | − | − | − | − | − | − | − | − | − | − |
| | 27923 | − | − | − | − | − | − | − | − | − | − |
| Naive | 26764 | − | − | + | + | − | − | − | − | − | − |
| | 28504 | − | − | + | + | + | + | + | + | − | − |
| | 29445 | − | − | + | + | + | + | + | + | + | − |

1. A recombinant fusion protein comprising
a first polypeptide comprising SEQ ID NO: 1 and
a second polypeptide comprising SEQ ID NO: 2; where the first polypeptide is located N-terminal relative to the second polypeptide.

2. An expression vector comprising a polynucleotide that encodes the recombinant fusion protein of claim 1 and a promoter, wherein the promoter is operably linked to the polynucleotide.

3. The expression vector of claim 2, where the polynucleotide that encodes the first polypeptide is derived from amplifying a nucleic acid fragment from dengue virus serotype 2 strain 16681 using a first oligonucleotide comprising SEQ ID NO: 3 and a second oligonucleotide comprising SEQ ID NO: 4.

4. A vaccine composition comprising the recombinant fusion protein of claim 1 and/or the expression vector of claim 2.

5. The vaccine composition of claim 4, further comprising an adjuvant.

6. The vaccine composition of claim 4 comprising the expression vector of claim 2, further comprising 1 μm diameter gold beads.

7. A method of generating an immune response to dengue virus serotype 2 in a subject, the method comprising administering an effective amount of the vaccine composition of claim 4 to the subject.

8. The method of claim 7 wherein the immune response is a protective immune response

9. The method of claim 7 where the vaccine composition of claim 4 comprises the recombinant fusion protein of claim 1 and where the pharmaceutical composition is administered intramuscularly.

10. The method of claim 7 where the vaccine composition of claim 4 comprises the expression vector of claim 2 and where the pharmaceutical composition is administered intradermally.

11. The method of claim 7 comprising administering the pharmaceutical composition comprising the recombinant fusion protein of claim 1 and administering the pharmaceutical composition comprising the expression vector of claim 2 to the subject.

12. The method of claim 11 where the effective amount of the recombinant fusion protein of claim 1 is between 65 and 95 μg/kg and the effective amount of the expression vector of claim 2 is between 5 μg/kg and 7 μg/kg.

13. The method of claim 11 where the pharmaceutical composition comprising the recombinant fusion protein of claim 1 and the pharmaceutical composition comprising the expression vector of claim 2 are administered on the same day in a first joint administration.

14. The method of claim 13 where the pharmaceutical composition comprising the recombinant fusion protein of claim 1 and the pharmaceutical composition comprising the expression vector of claim 2 are administered on the same day in a second joint administration and where the second joint administration is five weeks following the first joint administration.

15. The method of claim 13 where the pharmaceutical composition comprising the recombinant fusion protein of claim 1 and the pharmaceutical composition comprising the expression vector of claim 2 are administered on the same day in a third joint administration and where the third joint administration is 12 weeks following the first joint administration.
